(19) United States
(12) Patent Application Publication
(10) Pub. No.: US 2008/0188948 A1
(43) Pub. Date: Aug. 7, 2008

(54) LINER SYSTEM AND LINER FOR PROSTHETICS AND METHOD FOR USING AND MAKING

(76) Inventor: Terry J. Flatt, Plattsburgh, NY (US)

Correspondence Address: SCHMEISER, OLSEN & WATTS, 22 CENTURY HILL DRIVE, SUITE 302, LATHAM, NY, 12110, US

(21) Appl. No.: 11/671,091

(22) Filed: Feb. 5, 2007

(51) Int. Cl. A61F 2/80; A61F 2/60; B29C 33/40; B32B 37/06; B32B 37/08; B32B 37/10

(52) U.S. Cl. 623/36; 623/33; 264/222; 264/511

(57) ABSTRACT

A prosthetic limb socket liner system is provided. A compressible foam insert has a substantially cylindrical hollow interior which is defined by an interior insert surface and an opening. The opening and the hollow interior are configured to accept a substantial portion of a residual limb. A laminar pocket substantially covers the insert. The laminar pocket includes an elastomeric fabric layer which defines a concave interior, another elastomeric fabric layer which defines an exterior of the laminar pocket, and a flexible polymer layer between the two elastomeric fabric layers and bonded to the elastomeric fabric layers. The pocket has an open proximal end, a closed distal end. The open end and the concave interior are configured to accept a substantial portion of the compressible foam insert. The exterior of the laminar pocket is configured to fit into a socket of a prosthetic limb.

### FIELD OF THE INVENTION

The present invention relates generally to liner systems for use in a prosthetic limb and method for using and making the same.

### BACKGROUND OF THE INVENTION

Prosthetic limbs for amputees and limb deficient individuals require an interface between the residual limb and attachment point, or socket, of the prosthetic device in order to provide proper function. A user may place considerable force against a prosthetic limb such as during walking. Pressure and friction from the wall of the socket on the residual limb can severely damage soft tissue and skin. A cushioned liner between the residual limb and the rigid socket of the prosthetic limb into which it is fitted is needed to protect the residual limb from damage. There exists a need for a liner for a prosthetic limb that provides cushioning and support for a residual limb.

### SUMMARY OF THE INVENTION

A first aspect of the present invention provides a prosthetic limb socket liner system comprising:

a compressible foam insert, said insert comprising an opening and a substantially cylindrical hollow interior defined by an interior insert surface, said opening and said hollow interior configured to accept a substantial portion of a residual limb; and

a laminar pocket substantially covering said foam insert, said pocket comprising an open proximal end and a closed distal end, said open proximal end and said concave interior being configured to accept a substantial portion of said compressible foam insert, said exterior being configured to substantially fit into a socket of a prosthetic limb.

A second aspect of the present invention provides a method for cushioning a residual limb in a prosthetic socket comprising:

providing a compressible foam insert, said insert comprising a substantially cylindrical hollow interior defined by an interior insert surface and an opening, said opening and said hollow interior configured to accept a substantial portion of a residual limb;

inserting a distal end of said residual limb through said opening and into said hollow interior such that said limb substantially fills said hollow interior;

providing a socket of a prosthetic limb, said socket comprising a substantially concave interior and opening wherein said opening and said concave interior are configured to accept a substantial portion of said compressible foam insert, wherein said concave interior is defined by a concave laminar pocket; and

inserting said distal end of said residual limb with said foam insert into said concave interior of said socket of said prosthetic limb, such that said foam insert with said residual limb substantially fills said concave interior.

A third aspect of the present invention provides a method for forming a prosthetic socket liner comprising:

forming a first negative casting of a residual limb;

forming a first model of said residual limb from said first negative casting of said residual limb, said first model having an exterior surface, wherein said exterior surface substantially conforms to an exterior surface of said residual limb;

forming a first liner from said first model of said residual limb, said first liner having an opening and a first hollow interior substantially conforming to an exterior surface of said first model of said residual limb, wherein said first liner comprises a first material;

donning said first liner over said residual limb, wherein said residual limb substantially fills said first hollow interior;

forming a second negative casting, wherein said second negative casting is a casting of said first liner while said first liner is donned over said residual limb;

forming a second model of said second negative casting, said second model having an exterior surface substantially conforming to an exterior surface of said second negative casting;

forming a second liner from said second model, said second liner having an opening, an exterior defined by an exterior surface, and a second hollow interior defined by a second interior surface, said second interior surface substantially conforming to an exterior surface of said second model, wherein said second liner comprises a second material; and

adhering cover layers to said second interior surface and said second exterior outside surface of said second liner.

### BRIEF DESCRIPTION OF THE DRAWINGS

Various embodiments of the present invention will be described in detail, with reference to the following figures, wherein like designation denote like members wherein:

FIG. 1 is an illustration of an embodiment of a socket liner system comprising a flexible laminar pocket and a compressible foam insert, in accordance with embodiments of the present invention;

FIG. 2 is an illustration of a cross-sectional view of an embodiment of a socket liner system comprising a flexible laminar pocket and compressible foam insert, in accordance with embodiments of the present invention;

FIG. 3 is an illustration of a cross-sectional view of a distal end of a residual limb being positioned inside the socket liner system of FIG. 2, in accordance with embodiments of the present invention;

FIG. 4 is an illustration of a cross-sectional view of an embodiment of a socket liner system comprising a locking device, in accordance with embodiments of the present invention;

FIG. 5 is an illustration of an embodiment of a socket liner system comprising a compressible foam insert, a prosthetic socket, and a liner layer bonded to an interior surface of the socket, in accordance with the embodiments of the present invention;

FIG. 6 is a flow chart illustrating an embodiment of a method for forming a prosthetic socket liner system, in accordance with embodiments of the present invention;

FIG. 7 is a flow chart illustrating an embodiment of a method for forming a test socket to determine the fit of the liner system of FIG. 6, in accordance with the embodiments of the present invention.

The features of the invention are set forth in the appended claims. The invention itself, however, may be generally understood by reference to the following detailed description of illustrative embodiments when read in conjunction with the accompanying drawings.

### DETAILED DESCRIPTION OF THE INVENTION

Although certain specific embodiments of the present invention will be shown and described in detail, it should be understood that generalizations and various changes and modifications may be made without departing from the scope of the appended claims. The scope of the present invention will in no way be limited to the number of constituting components, the materials thereof, the shapes thereof, the relative arrangement thereof, etc. Some features of the present invention are illustrated in detail in the accompanying drawings, wherein like reference numerals refer to like elements throughout the drawings. Although the drawings are intended to illustrate an embodiment of the present invention, the drawings are not necessarily drawn to scale.

A need for the present invention may relate to the loss of soft tissue of an amputee's residual limb with liner systems for prosthetic limbs which may employ an individual foam or silicone gel elastomer liner, for example. The loss of soft tissue may be a major contributor to the breakdown of the skin of the residual limb due to the tremendous pressures exerted against the surface of the residual limb, which may lead to fluids being forced out of the tissues, and may lead to cyst growth and infection. The use of a multi-layered system as described within the scope of the present invention may prevent or limit loss or damage to tissues and related injuries.

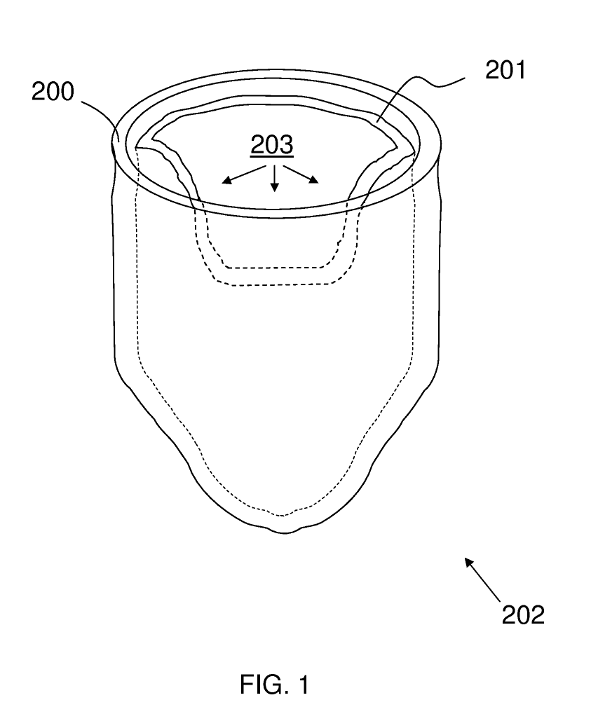

FIG. 1 is an illustration of an embodiment of a socket liner system 202 comprising a flexible laminar pocket 200 and a compressible foam insert 201. The laminar pocket 200 may be configured to substantially cover the foam insert 201. The compressible foam insert 201 may have a cut-away section 203 to allow for flexibility when a residual limb is inserted into the insert 201 during use.

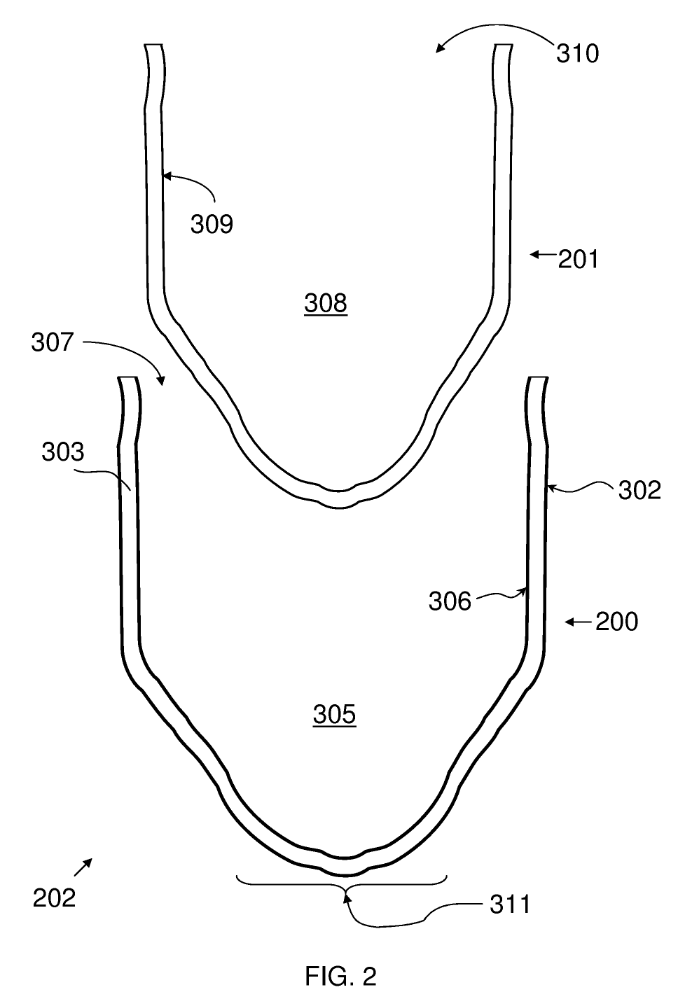

FIG. 2 is an illustration of a cross-sectional view of an embodiment of two separated sections of an embodiment of a socket liner system 202 comprising a flexible laminar pocket 200 and compressible foam insert 201. The laminar pocket 200 may be configured to substantially cover the foam insert 201, where the laminar pocket 200 may comprise a concave interior 305, an open proximal end 307, and a closed distal end 311. The open proximal end 307 and the concave interior 305 may be configured to accept a substantial portion of the compressible foam insert 201. The laminar pocket may further comprise a first elastomeric fabric layer 306 defining the concave interior 305, a second elastomeric fabric layer 302 defining an exterior of the laminar pocket, and a flexible polymer layer 303 disposed between the first elastomeric fabric layer 306 and the second elastomeric fabric layer 302. The flexible polymer layer 303 may be bonded to the first elastomeric fabric layer 306 and to the second elastomeric fabric layer 302. The exterior of the laminar pocket 200 may be configured to substantially fit into a socket of a prosthetic limb.

The compressible foam insert 201 may have a substantially cylindrical hollow interior 308 defined by an interior insert surface 309 and opening 310, where the opening 310 and the substantially cylindrical hollow interior 308 may be configured to accept a substantial portion of a residual limb. Such a configuration may be achieved, for example, by forming a casting or model of a user's residual limb and molding the foam insert from the casting to provide a substantially precise fit for the user. A precise socket liner fit may assist with eliminating gaps between the liner and the user's residual limb, wherein such gaps may be exacerbated by residual limbs which are invaginated or bony and have protruding areas. Gaps between the residual limb and the liner can cause tissue damage to the residual limb due to shear stresses and shock during use of a prosthetic. The compressible foam insert 201 may provide cushioning to absorb shock to the residual limb while using a prosthetic limb, and may have a sufficiently low percent compression set such that extended use does not eliminate the cushioning benefits. Processes such as thermoforming and compression molding, for example, may be used to mold the insert 201 if it is comprised of thermoplastics. Thermoset materials may be cast from a reaction of their precursor materials to form the insert 201.

The first elastomeric fabric layer 306 and second elastomeric fabric layer 302 may be made from nylon, cotton, polyester, and/or the like, or combinations of these. For instance, the first elastomeric fabric layer 306 and second elastomeric fabric layer 302 may be a woven blend of nylon, cotton, or polyester, with a fiber-forming substance which may be a long chain synthetic polymer comprised of at least 85 percent of a segmented polyurethane, such as spandex. Examples of spandex may include LYCRA and ELASPAN. Other synthetic and natural fibers may be suitable for blending with the long chain synthetic polymer comprised of at least 85 percent of a segmented polyurethane, to provide an acceptable elastomeric fabric for the elastomeric fabric layer. The first elastomeric fabric layer 306 may further comprise a moisture-wicking synthetic fabric design of nylon or polyester, such as COOLMAX. The flexible polymer layer 303 may add axial rigidity to the socket liner 202 to aid in donning, and may allow radial flexibility for fitting the liner 202 over contours of a residual limb which may protrude.

The flexible polymer layer 303 may comprise a material such as polyurethane, silicone, silicone gel elastomer, thermoplastic elastomer, combinations of these, and the like, to provide suitable flexibility for a socket liner system 202 using this material. The flexible polymer layer 303 may have a thickness in a range from about 3 mm to about 6 mm. An acceptable Durometer hardness range for the flexible polymer layer 303 may be in the range from about 20 to about 70 on the Shore A hardness scale, or may be in the range from about 5 to about 70 on the Shore 00 hardness scale.

The compressible foam insert 201 may comprise a material such as polyurethane, polyethylene, poly(vinyl chloride) (PVC) closed cell foam, a viscoelestic polyurethane foam such as ISOFORM, a closed-cell crosslinked polyethylene foam such as PELITE, ALIPLAST or PLASTAZOTE, PVC-nitrile rubber foam such as ENSOLITE, a combination of these materials, and the like. The foam insert may be sufficiently compressible to absorb shock during use and sufficiently resilient to substantially return to its original thickness (releasing any compression set) when weight or pressure from an artificial limb and/or socket is removed.

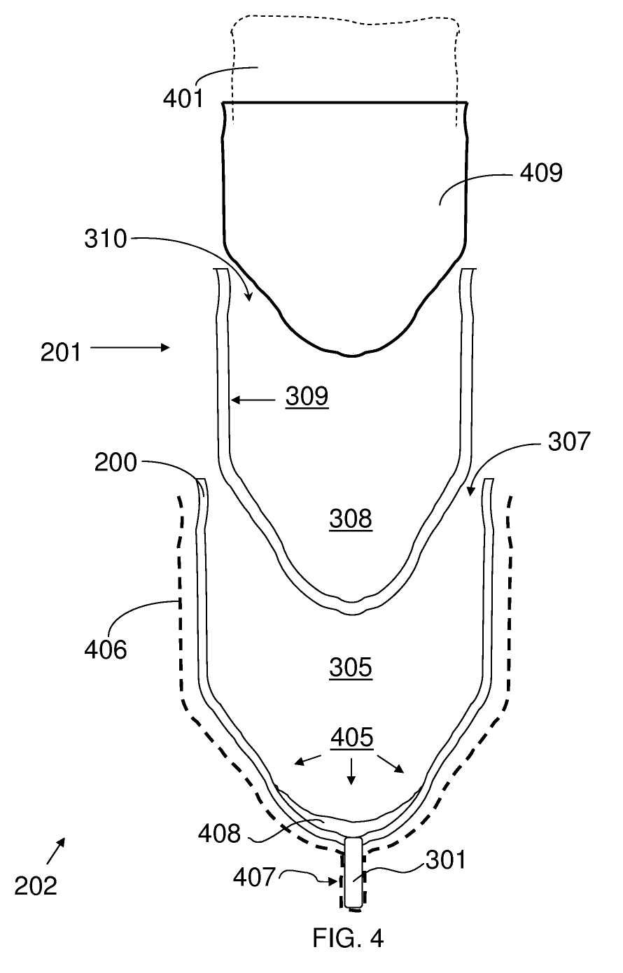

The first elastomeric fabric layer 306 may provide a comfortable interior surface for a user where a portion of the laminar pocket 200 may be exposed to the skin of the residual limb, such as when the foam insert 201 has a cut-away section (203 in FIG. 1) to allow for flexibility, for example. Materials such as a woven blend of nylon, cotton, or polyester, with a fiber-forming substance which may be a long chain synthetic polymer comprised of at least 85 percent of a segmented polyurethane, may have sufficient elastic properties to accommodate movement and stretching of the laminar pocket 200 as it is fitted into the prosthetic socket (such as socket 406 shown in FIG. 4), and when a foam insert 201 worn on a residual limb is inserted inside the laminar pocket's concave interior. The fabric may provide a soft breathable surface, which may not “tack up” against the skin of the user and cause tissue damage due to friction, moisture, and shear forces during use. The use of a moisture-wicking synthetic fabric design of nylon or polyester, such as COOLMAX may allow for the elimination of moisture associated with perspiration and may reduce associated skin irritation and tissue softening.

The materials used within the scope of the present invention may contain mineral oil, vitamins, or a combination of these. Such additives may be absorbed by the skin of the user and may sooth and protect the tissue of the residual limb of the user.

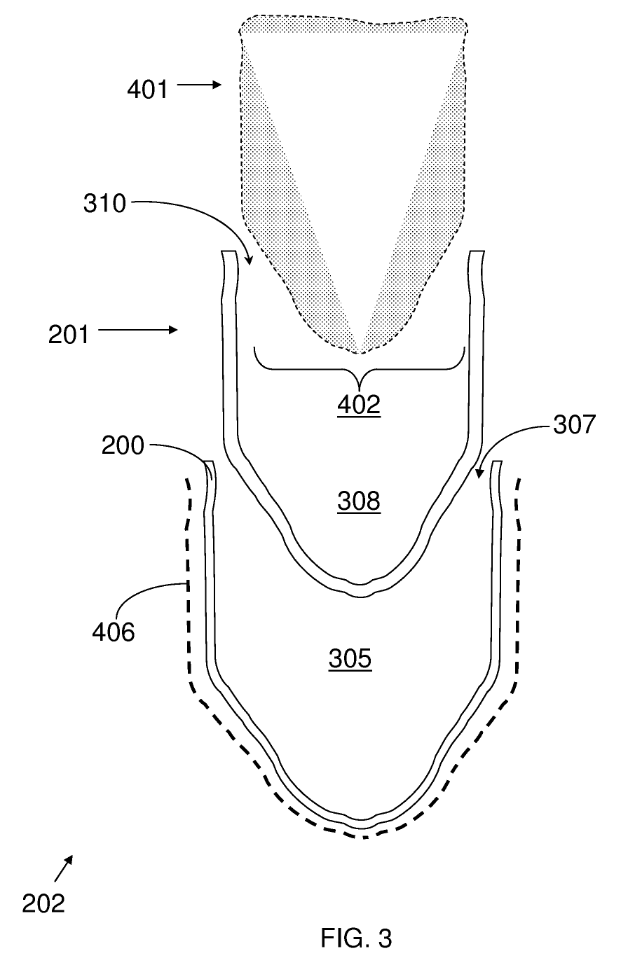

FIG. 3 is an illustration of a cross-sectional view of a distal end 402 of a residual limb 401 being positioned inside the socket liner system 202 of FIG. 2. A user may insert the distal end 402 of a residual limb 401 through the opening 310 of the compressible foam insert 201 and into the concave interior 308 of the compressible foam insert 201 such that the residual limb 401 substantially fills the concave interior 308. The distal end 311 of the laminar pocket 200 may be inserted into the socket 406 of a prosthetic limb, such that the laminar pocket 200 substantially fills the socket 406. After inserting the distal end 311 of the laminar pocket 200 into the socket 406, the compressible foam insert 201 with said residual limb 401 may be inserted through the open proximal end 307 of the laminar pocket 200 such that the insert 201 with the residual limb 401 substantially fill the concave interior 305 of the laminar pocket 200.

The socket liner system 202 may further comprise a woven fabric sock 409 as in FIG. 4, wherein the woven fabric sock 409 may be worn directly over the residual limb 401. A user may insert the residual limb 401 into the woven fabric sock 409 prior to inserting the residual limb 401 into the compressible foam insert 201, such that the woven fabric sock 409 is disposed between the residual limb 401 and the compressible foam insert 201.

The woven fabric sock 409 may be impregnated or infused with an elastomer by infusing the material with the elastomer while it is in a molten state, while it is being formed from its reactant materials, while it is dissolved in a suitable solvent, a combination of these, or any other means for infusing the elastomer into the woven fabric of the sock 409. When the liner system 202 is in use, the infused elastomer within the woven fabric of the sock 409 may provide a flexible cushion as it may be compressed into and fill small gaps remaining between the compressible foam insert 201 and prosthetic socket 406. Gaps between the insert 201 and socket 406 may be a source of jarring or shock to a user, such as during walking with a prosthetic limb on a lower extremity such as a foot or leg, or when pushing or lifting with an upper extremity prosthetic limb. Filling such gaps with a shock absorbing elastomer may alleviate the jarring and prevent damage to the residual limb and associated tissue.

The woven fabric sock 409 may comprise cotton, nylon, linen, polypropylene, neoprene, rayon, silk, wool, cellulose acetate, acrylic, polylactide, aromatic polyamide, polyester, a combination of these, and the like. Moreover, the woven fabric sock 409 may be impregnated or infused with an elastomer such as a thermoplastic elastomer, an elastomeric gel, natural rubber, latex, silicone, neoprene, butyl rubber, polyurethane, a block copolymer, a combination of these, or any other polymer having elastomeric properties. The infused elastomer may be of a sufficient durometer, molecular weight, and crosslink density such that it is permanently infused within the woven fabric sock 104 and allows elastic flexibility and cushioned support. An acceptable Durometer hardness range for the elastomer infused in the woven fabric layer 104 may be in the range from about 5 to about 75 on the Shore 00 hardness scale.

FIG. 4 is an illustration of a cross-sectional view of an embodiment of a socket liner system 202 comprising a locking device 301, such as a locking pin for example, which may releasably secure the laminar pocket 200 to a corresponding attachment point 407, such as an opening or slot, in a prosthetic limb socket 406. A suitable mechanism may be used on the locking device 301 of the liner 202 such as an interlocking configuration having corresponding features in the slot or opening 407, to allow for securely attaching and the laminar pocket 200 to the prosthetic socket 406, and then easily releasing the laminar pocket 200 when desired by the user. Furthermore, the locking device 301 may comprise a locking pin, a threaded fastener, a barbed pin, a partially compressible polymeric pin, combinations of these, or the like. The locking device 301 may be securely attached to a distal cup 408 located and attached to the distal end 311 of the laminar pocket 200. The distal cup 408 may be comprised of rigid plastic to securely anchor the locking device 301 to the distal end 405 of the flexible liner 200. A user may releasably secure the locking device 301 to the corresponding attachment point 407 in the socket 406 of the prosthetic limb after inserting the laminar pocket 200 into the socket 406 of said prosthetic limb.

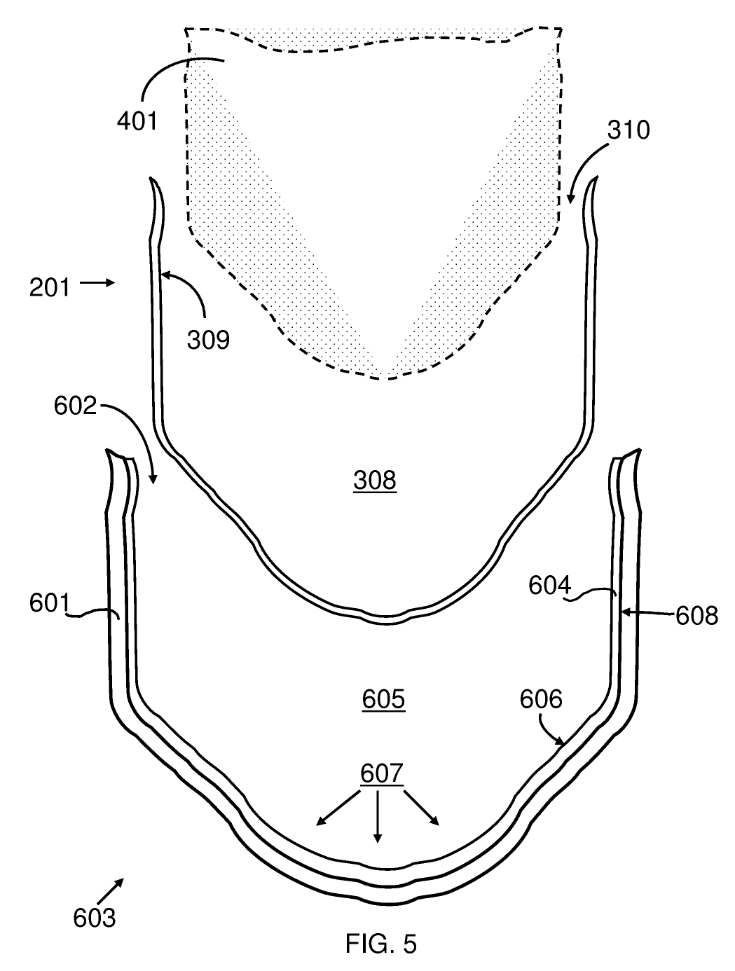

FIG. 5 is an illustration of an embodiment of a socket liner system 603 comprising a compressible foam insert 201, a laminar pocket 604, and a socket 601 of a prosthetic limb. The compressible foam insert 201 may comprise an opening 310 and a substantially cylindrical hollow interior 308 defined by an interior insert surface 309. The opening 310 and the hollow interior 308 may be configured to accept a substantial portion of a residual limb 401. The laminar pocket 604 may be configured to substantially cover the foam insert 201 when the insert 201 is inserted into a substantially concave interior 605 of the laminar pocket 604. The laminar pocket 201 may comprise an open proximal end 602, a closed distal end 607, a substantially concave interior 605 defined by a flexible cover layer 606, and a flexible polymer layer 604. In addition, the flexible cover layer 606 may be disposed between the foam insert 201 and the flexible polymer layer 604. The flexible polymer layer 604 may be disposed between and bonded to both the flexible cover layer 606 and an interior surface 608 of the prosthetic socket 601, where the flexible cover layer 606 may substantially cover the flexible polymer layer 601. The open proximal end 602 and the concave interior 605 may be configured to accept a substantial portion of the compressible foam insert 201, where the flexible polymer layer 604 may be configured to substantially fit into the socket 601 of a prosthetic limb.

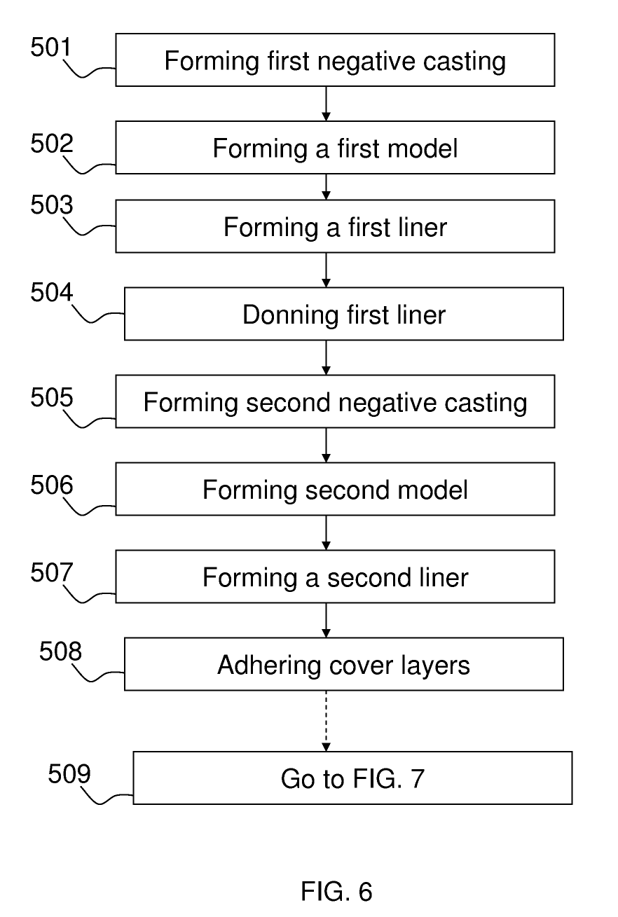

FIG. 6 is a flow chart illustrating an embodiment of a method for forming a prosthetic socket liner system. Step 501 provides forming a first negative casting of a residual limb, which may be formed by forming a plaster cast over the residual limb and then removing the cast from the limb to produce the negative casting, for example. Step 502 provides forming a first model of the residual from the first negative casting of step 501. This may be accomplished, for example, using plaster of Paris by pouring it into the negative casting and removing the newly formed model after the plaster has hardened to produce a substantially identical model of the amputee's residual limb. Casting materials such as plaster of Paris, lime plaster, earthen plaster, cement plaster, silicone, thermoplastics, wax, combinations of these, and the like may be used to form the castings and models within the scope of the present invention.

Step 503 provides forming a first liner from the first model of the residual limb. The first liner may be formed from a material such as polyurethane, polyethylene, poly(vinyl chloride) (PVC) closed cell foam, a viscoelestic polyurethane foam such as ISOFORM, a closed-cell crosslinked polyethylene foam such as PELITE, ALIPLAST or PLASTAZOTE, PVC-nitrile rubber foam such as ENSOLITE, a combination of these materials, and the like. The first liner material may be sufficiently compressible to absorb shock during use, and sufficiently resilient to substantially return to its original thickness (releasing any compression set) when weight or pressure from an artificial limb and/or socket is removed. Processes such as thermoforming and compression molding, for example, may be used to form the first liner if it is comprised of a thermoplastic. Thermoset materials may be formed from a reaction of their precursor materials to form the first liner.

Step 504 provides donning the first liner over the residual limb such that the residual limb substantially fills the interior of the first liner. In step 505, a second negative casting may be formed from the first liner while it is worn over the residual limb. Forming the casting may be accomplished in a similar fashion to step 501.

Step 506 provides forming a second model of the second negative casting. The second model may have an exterior surface that conforms to the exterior surface of the second negative casting.

Step 507 provides forming a second liner from the second model, such that the second liner may have an opening, an exterior surface, and a hollow interior. The hollow interior of the second liner may have an interior surface that conforms to the exterior surface of the second model. Since the second model may conform to the exterior surface of the first liner while being worn on the residual limb, the interior of the second liner may be configured to accept the first liner. The second liner may comprise a flexible polymer layer which may be comprised of a material such as polyurethane, silicone, a thermoplastic elastomer, silicone rubber, latex, a combination of these, and the like. An acceptable Durometer hardness range for the second liner 303 may be in the range from about 20 to about 70 on the Shore A hardness scale, or may be in the range from about 5 to about 70 on the Shore 00 hardness scale.

In Step 508, flexible cover layers may be adhered to the interior and exterior surfaces of the second liner, where the cover layers may substantially cover the interior and exterior surfaces of the second liner. The cover layers may be elastomeric fabric, an elastomer, copolymer, or combinations thereof. The cover layers may be adhered using an adhesive such as a solvent-based adhesive, water-based adhesive, hot-melt, epoxy, and the like which may bond to both surfaces. The adhesion may utilize a thermal process, or any other means for adhering the cover layers to the liner surfaces. The cover layers and second liner material may be bonded together during their formation from their reactant materials, such as in an example where the second liner is a urethane layer which is bonded directly onto either of the cover layers during the polymerization reaction to form the urethane layer. The layers may be bonded by melting or during a thermoforming process, such as with a thermoplastic elastomer (TPE) as the second liner material. In such a case, the TPE may melt, flow, and physically attach to the cover layer. The cover layers may be placed over the surface of the second model prior to forming the second liner, which may provide a means for adhering the cover layers to the interior of the second liner if the material comprising the second liner is sufficiently melted to adhere to the cover layers. Adhesive may be applied to the cover layers after they are placed on the second model surface, and then the second liner may be formed onto the adhesive layer. Prior to adhering, the surfaces to be adhered may be pretreated to promote adhesion, such as with a plasma, corona, flame, ozone, chemical adhesion promoter, or similar surface activation technique.

In another embodiment within the scope of the present invention, the second liner may be adhered to the interior surface of a prosthetic socket of a prosthetic limb, using the adhesive and adhering methods above. In such a case, the cover layer on the exterior surface of the second liner may be omitted from the second liner and the flexible polymer layer may be adhered directly to the interior surface of the socket. The socket may comprise polymer laminates with materials such as fiberglass, nylon, polyester fiber such as the polyethylene terephthalate fiber DACRON, carbon fiber, polyaramid fiber such as KEVLAR, ultra high molecular weight polyethylene fiber such as SPECTRA, polypropylene, epoxy resin, epoxy adhesive, combinations thereof, or any other material which may provide sufficient strength to the prosthetic limb socket so that the limb may withstand the forces associated with use.

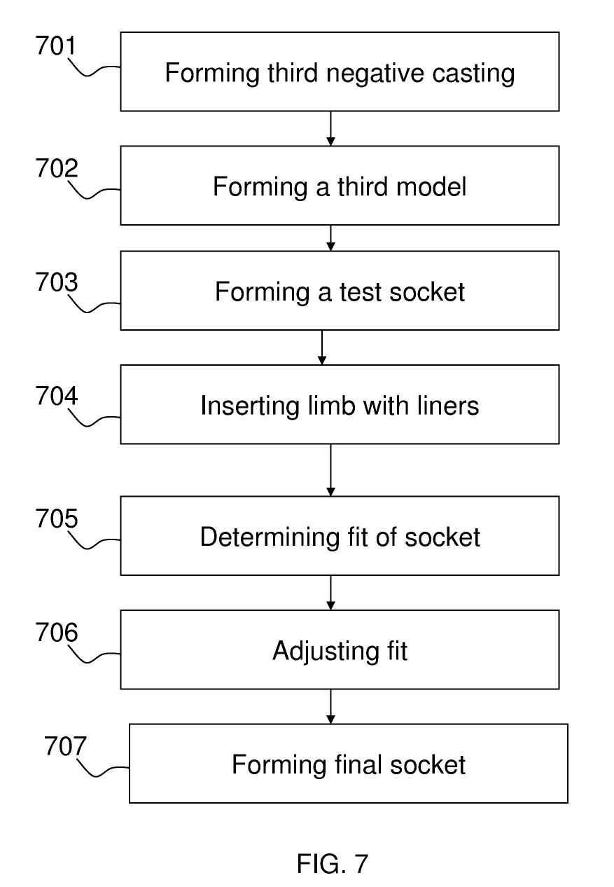

FIG. 7 is a flow chart illustrating an embodiment of a method for forming a test socket to determine the fit of the liner system of FIG. 6 prior to forming a final prosthetic socket (see step 509 in FIG. 6). A test socket may be formed to determine the fit of the liner system and to allow the user to provide any small adjustments needed prior to making a final socket.

In step 701, a third negative casting may be made of the residual limb donned with the first and second liners using methods such as those described in step 501. In step 702, a third model may be made from the third negative casting using methods describe above in step 503, where the third model has an exterior surface that substantially conforms to the exterior surface of the residual limb donned with the first and second liners. In step 703, the test socket is formed from the third model by thermoforming a socket from materials such as polyacrylics, polyolefins, thermoplastics, combinations thereof, and the like. In step 704 the residual limb donned with the first and second liners and then inserted into the test socket. The fit of the test socket may be determined in step 705 by noting the user's comfort and control of the prosthetic limb during use. In step 706 the fit may be adjusted by the removal or addition of material to the outer surface of the liner, or the inner surface of the test socket. The final socket may formed in step 707 by making a socket model from the interior of the test socket, and forming the final socket from the socket model.

The socket model may be made using the methods above for the first, second, and third models, such as with plaster of Paris for example. The final socket may comprise polymer laminates with materials such as fiberglass, nylon, carbon fiber, polyaramid fiber such as KEVLAR, ultra high molecular weight polyethylene fiber such as SPECTRA, polypropylene, epoxy resin, epoxy adhesive, combinations thereof, or any other material which may provide sufficient strength to the prosthetic limb socket so that the limb may withstand the forces associated with use.

The foregoing description of the present invention has been presented for purposes of illustration and description. It is not intended to be exhaustive or to limit the invention to the precise form disclosed, and many modifications and variations are possible in light of the above teaching. Such modifications and variations that may be apparent to a person skilled in the art are intended to be included within the scope of this invention as defined by the accompanying claims.

What is claimed is:

1. A prosthetic limb socket liner system comprising:
a compressible foam insert, said insert comprising an opening and a substantially cylindrical hollow interior defined by an interior insert surface, said opening and said hollow interior configured to accept a substantial portion of a residual limb; and
a laminar pocket substantially covering said foam insert, said pocket comprising an open proximal end and a closed distal end, said open proximal end and said concave interior being configured to accept a substantial portion of said compressible foam insert, said exterior being configured to substantially fit into a socket of a prosthetic limb.

2. The socket liner system of claim 1, wherein said laminar pocket further comprises a first elastomeric fabric layer defining a concave interior, a second elastomeric fabric layer defining an exterior of said laminar pocket, and a flexible polymer layer bonded to said first elastomeric fabric layer and said second elastomeric fabric layer and disposed there between, wherein said first and second elastomeric fabric layers are comprised of a woven blend of polyurethane with material selected from the group consisting of nylon, cotton, polyester, and combinations thereof.

3. The socket liner system of claim 2, wherein said polyurethane is spandex.

4. The socket liner system of claim 1, wherein said flexible polymer layer comprises a material selected from the group consisting of polyurethane, silicone, silicone gel elastomer, thermoplastic elastomer, and combinations thereof.

5. The socket liner system of claim 1, wherein said compressible foam comprises a material selected from the group consisting of polyurethane, polyethylene, poly(vinyl chloride) closed cell foam, viscoelestic polyurethane, closed-cell crosslinked polyethylene, PVC-nitrile rubber, and combinations thereof.

6. The socket liner system of claim 1, wherein said prosthetic limb is selected from the group consisting of a lower extremity prosthetic limb and an upper extremity prosthetic limb.

7. The socket liner system of claim 1, further comprising a locking device connected to said distal end of said laminar pocket for releasably securing said pocket to a corresponding attachment point in said socket of said prosthetic limb.

8. The socket liner system of claim 1, further comprising a woven fabric sock configured to be worn directly over a residual limb, said woven fabric having infused therein an elastomer.

9. The socket liner system of claim 8, wherein said woven fabric comprises material selected from the group consisting of spandex, cotton, nylon, linen, polypropylene, neoprene, rayon, silk, wool, cellulose acetate, acrylic, polylactide, aromatic polyamide, polyester, and combinations thereof.

10. The socket liner system of claim 8, wherein said elastomer is selected from the group consisting of thermoplastic elastomer, elastomeric gel, natural rubber, latex, silicone, neoprene, butyl rubber, polyurethane, a block copolymer, and combinations thereof.

11. A method for cushioning a residual limb in a prosthetic socket comprising:
providing a compressible foam insert, said insert comprising a substantially cylindrical hollow interior defined by an interior insert surface and an opening, said opening and said hollow interior configured to accept a substantial portion of a residual limb;
inserting a distal end of said residual limb through said opening and into said hollow interior such that said limb substantially fills said hollow interior;
providing a socket of a prosthetic limb, said socket comprising a substantially concave interior and opening wherein said opening and said concave interior are configured to accept a substantial portion of said compressible foam insert, wherein said concave interior is defined by a concave laminar pocket; and
inserting said distal end of said residual limb with said foam insert into said concave interior of said socket of said prosthetic limb, such that said foam insert with said residual limb substantially fills said concave interior.

12. The method of claim 11, wherein said socket is comprised of materials selected from the group consisting of polypropylene, fiberglass, nylon, polyester fiber, carbon fiber, polyaramid fiber, ultra high molecular weight polyethylene fiber, epoxy resin, epoxy adhesive, and combinations thereof.

13. The method of claim 11, wherein said pocket comprises a flexible polymer layer bonded to an interior surface of said socket and a flexible cover layer substantially covering and bonded to said flexible polymer layer such that said flexible polymer is disposed between said interior surface and said cover layer, wherein said flexible cover layer comprises material selected from the group consisting of elastomeric fabric, elastomer, copolymer, or combinations thereof.

14. The method of claim 11, wherein said compressible foam comprises a material selected from the group consisting of polyurethane, polyethylene, poly(vinyl chloride) closed cell foam, viscoelestic polyurethane, closed-cell crosslinked polyethylene, PVC-nitrile rubber, and combinations thereof.

15. The method of claim 14, wherein said material contains mineral oil, vitamins, or a combination thereof.

16. A method for forming a prosthetic socket liner comprising:
forming a first negative casting of a residual limb;
forming a first model of said residual limb from said first negative casting of said residual limb, said first model having an exterior surface, wherein said exterior surface substantially conforms to an exterior surface of said residual limb;
forming a first liner from said first model of said residual limb, said first liner having an opening and a first hollow interior substantially conforming to an exterior surface of said first model of said residual limb, wherein said first liner comprises a first material;
donning said first liner over said residual limb, wherein said residual limb substantially fills said first hollow interior;
forming a second negative casting, wherein said second negative casting is a casting of said first liner while said first liner is donned over said residual limb;
forming a second model of said second negative casting, said second model having an exterior surface substantially conforming to an exterior surface of said second negative casting;
forming a second liner from said second model, said second liner having an opening, an exterior defined by an exterior surface, and a second hollow interior defined by a second interior surface, said second interior surface substantially conforming to an exterior surface of said second model, wherein said second liner comprises a second material; and
adhering cover layers to said second interior surface and said second exterior outside surface of said second liner.

17. The method of claim 16, wherein said forming said first liner is a process selected from the group consisting of thermoforming, vacuum forming, and compression forming.

18. The method of claim 16, wherein said first material comprises a material selected from the group consisting of polyurethane, polyethylene, poly(vinyl chloride) closed cell foam, viscoelestic polyurethane, closed-cell crosslinked polyethylene, PVC-nitrile rubber, and combinations thereof.

19. The method of claim 16, wherein said second material is polyurethane, silicone, a thermoplastic elastomer, silicone rubber, latex, or combinations thereof.

20. The method of claim 16, wherein said cover layers comprise elastomeric fabric comprised of a woven blend of spandex with material selected from the group consisting of nylon, cotton, polyester, and combinations thereof.
